(19) United States
(12) Patent Application Publication
(10) Pub. No.: US 2012/0010705 A1
(43) Pub. Date: Jan. 12, 2012

(54) Light-Weight External Breast Prosthesis

(75) Inventors: Aldo A. Laghi, Clearwater, FL (US); Nathaniel Vint, Oldsmar, FL (US)

(73) Assignee: ALPS South IP, St. Petersburg, FL (US)

(21) Appl. No.: 12/832,393

(22) Filed: Jul. 8, 2010

(51) Int. Cl. A61F 2/52

(52) U.S. Cl. 623/7

(57) ABSTRACT

An external lightweight breast prosthesis comprising an elastomeric polyurethane skin filled with copolymer gel filler comprising a mixture of mineral oil, thermoplastic copolymer and glass microspheres and initially configured to approximate the shape of a natural breast wherein the breast prosthesis can be reconfigured to be customized to the individual wearer by subjecting the prosthesis to heating, reshaping, and followed by cooling until the prosthesis retains its new shape.

### FIELD OF THE INVENTION

The invention relates to an external breast prosthesis, and in particular to a breast prosthesis that can be reshaped or customized to the individual user.

### BACKGROUND OF THE INVENTION

External breast prostheses are artificial breast forms that can be worn after a surgery or other treatment in which the breast has been altered or removed. For example, external breast prostheses are available for women who have had a mastectomy or lumpectomy to remove breast cancer, and to those who have uneven or unequal sized breasts resulting from radiation, reconstruction procedures or birth defects.

Currently, most prosthetic breasts consist of a polyurethane film outer skin filled with a soft silicone, foamed silicone gel, or some other type of silicone elastomer usually containing any number of filler materials, herein referred to as silicone. The form is set in a particular shape as the silicone vulcanizes. This shape is permanent and is determined by the shape of the mold used to manufacture the part. The silicone is soft and relatively lightweight but does not provide any drape or movement that would be expected from an actual human breast. Prosthetic breasts presently on the market are also filled with such soft silicone materials mixed together with glass micro spheres to lighten the weight of the prosthesis.

Generally, silicone gel has become the most widely accepted material used in external breast prostheses, for the most part for its resilient properties. A prime example of this style of external breast prosthesis is disclosed in U.S. Pat. No. 4,019,209 issued to Spence. However, in the ensuing years, it became apparent that the weight of the gel is such that it produces undo strain on the mastectomy patient's back, resulting in side effects ranging from discomfort to painful back injuries. As a result, significant effort has been undertaken to develop lighter weight breast prostheses without losing the look, feel and behavior of a natural breast. One such improvement is disclosed in U.S. Pat. No. 4,380,569 issued to Shaw. As illustrated in FIG. 3, the external breast prosthesis includes an elastic covering layer 24 enclosing a silicone gel core 20 containing glass microspheres 22 with a back piece 26. Although this product provided lighter weight breast prostheses, it was at the expense of the natural look, feel and behavior of a natural breast. Another breast prosthesis designed to be light-weight but not at the expense of the natural look, feel and behavior is disclosed in U.S. Pat. No. 6,066,220 issued to Schneider-Nieskens. As illustrated in FIG. 1, Schneider-Nieskens developed an external breast prosthesis having an inner core 2 housed within cover layers 3 and 4. The cover layers are each comprised of two foils 5, 6, 7 and 8 made of thermoplastic polyurethane between which is a layer of standard silicone gel. The inner core 2 is enclosed within the cover layers and consists of a silicone compound mixed with lightweight fillers such as micro glass spheres.

Further efforts in the design an even lighter external breast prosthesis are disclosed in U.S. Pat. No. 5,902,335 and U.S. Published Application No. 2007/0293945. These type of breast prostheses included dual chambers, i.e., an outer chamber filled with regular silicone gel mixed with glass microspheres to reduce weight, and an inner chamber filled with just regular silicone gel.

It is noted that all of the above discussed breast prostheses are made of silicone gel. As previously discussed the silicone assumes the shape of the mold that it was formed in, and further shaping is not possible. Since no two women have the same residual tissue configuration, it is not ideal to have a breast form of the exact same shape for everyone. The use of silicone gel in the currently known products presents a limitation in developing an extremely lightweight breast prosthesis that retains the look, feel, and behavior of a natural breast.

Furthermore, lightweight silicone sometimes referred to as foamed or whipped silicone is very costly to produce. The manufacturers which use this material often must have it imported from Europe where the majority of it is manufactured. Or the breast forms themselves are made overseas where the silicone is supplied. Lead times and costs of the filler material can be considerable.

In light of the limitations of the currently known breast prostheses, there exists a tremendous need not only for an extremely lightweight breast prosthesis, but also one less expensive and one capable of being reconfigured or reshaped to be customized to the individual user. Applicant's invention, as discussed in greater detail hereinbelow, provides a solution to such drawbacks.

### BRIEF SUMMARY OF THE INVENTION

In view of the foregoing, it is an object of the present invention to provide an external breast prosthesis that is extremely lightweight.

It is another object of the present invention to provide an external breast prosthesis that is capable of being reconfigured or reshaped to be customized to the individual user.

It is another object of the present invention to provide an external breast prosthesis that is less expensive than other prior art breast prostheses.

It is another object of the present invention to provide an external breast prosthesis that is more exact to the natural breasts of the user.

It is another object of the present invention to provide an external breast prosthesis that closely mimics the natural feel of a natural breast.

It is another object of the present invention to provide an external breast prosthesis that closely mimics the natural behavior of a natural breast.

It is another object of the present invention to provide an external breast prosthesis that is comfortable to wear and will minimize strain on a user's spine.

To accomplish the above objectives, the external breast prosthesis of the present invention uses a polyurethane film outer skin filled with a different type of gel filler which consists of a mixture of white mineral oil, hydrogenated styrenic block copolymer, and glass micro spheres (for weight reduction). The combination of these ingredients will be referred to as copolymer gel filler from here forward. The copolymer gel filler provides a much more realistic feel than the silicone gel filler. In addition the drape and rheological properties are more consistent with natural human tissue.

The copolymer gel filler is a thermoplastic elastomer that can be reshaped with the application of heat. The breast prosthesis of the present invention could provide a considerable advantage over silicone gel filled breast prostheses with respect to customization. It is highly desirable to have a breast form that conforms to the shape of the residual tissue, both for quality of appearance and more importantly comfort. The tissue of a post operative chest cavity is highly sensitive for a considerable amount of time following the surgery so any addition to comfort level could greatly increase quality of life for the patient.

The copolymer gel filler used in the present invention is mostly comprised of mineral oil which is an abundant commodity. Thus, the cost of processing the copolymer gel filler of the present invention is considerable less that the cost of any silicone gel filler available. Also, the copolymer gel of the present invention has a density of 0.87 gram/cubic centimeter, which is considerably lower than silicone at 0.99 grams/cubic centimeter. These are densities before any filler or glass bubbles are added.

### BRIEF DESCRIPTION OF THE DRAWINGS

FIG. 1 illustrates a perspective view of the external breast prosthesis of the present invention.

FIG. 2 illustrates a cross-sectional view of the external breast prosthesis of the present invention.

### DETAILED DESCRIPTION OF THE INVENTION

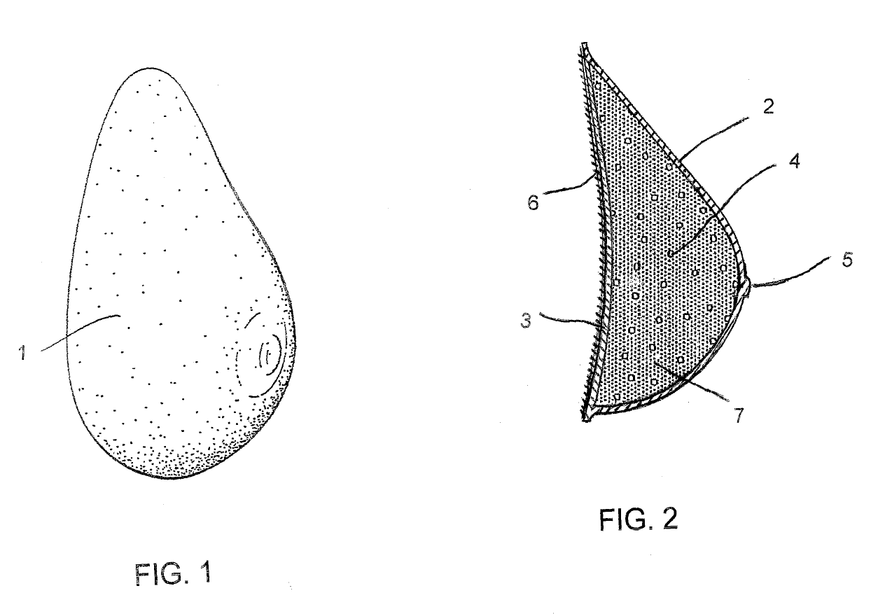

Referring to FIG. 1, a perspective view of the lightweight breast prosthesis (1) of the present invention is illustrated. Although the perspective view is taken from a side angle, the breast prosthesis is symmetrical about its central axis affording its use to either the left or right chest of the user.

Referring to FIG. 2, a cross-sectional view of the breast prosthesis of the present invention is illustrated. The breast prosthesis includes front skin (2) and rear skin (3) comprised of polyurethane films and initially formed to approximate the shape of a natural breast by any conventional molding technique. The front skin (2) is secured at its peripheral edge to the peripheral edge to rear skin (3) such as by an adhesive or by any conventional heat sealing technique. The front skin also has nipple (5) formed therein during the molding process. Contained within the front and rear skins is a copolymer gel filler (7). The copolymer gel filler consists of a mixture of white mineral oil, hydrogenated styrenic block copolymer comprised of a SEPS type: polystyrene-b-poly(ethylene/propylene)-b-polystyrene, of a SEPS type: polystyrene-b-poly(ethylene/butylene)-b-polystyrene, of a SEEPS type: polystyrene-b-poly(ethylene-ethylene/propylene)-b-polystyrene, or a mixture of any of the above copolymers, and glass micro spheres. As illustrated in FIG. 2, the glass microspheres (4) are added mainly to reduce the weight of the copolymer. In the preferred embodiment, the mixture consists of approximately 89% Kaydol® oil, 3.5% Septon® 2006 SEPS, 0.2% Septon® 4033 SEEPS, and 7.3% glass micro spheres. However, this ratio is not concrete and could consist of 80% to 93% mineral oil, 2% to 12% copolymer, and 0% to 15% glass micro spheres depending on the desired feel of the final product. Also, although in the preferred embodiment, the block copolymer consists of Septon® 2006 or 4033, although others may be used.

Other types of block copolymers may include a triblock copolymer or combinations thereof, such as a hydrogenated poly(styrene-b-isoprene), a hydrogenated poly(styrene-b-isoprene-b-styrene), a hydrogenated poly(styrene-b-butadiene-b-styrene), a hydrogenated poly(styrene-b-isoprene/butadiene-b-styrene), or combinations thereof. Also, a polystyrene-b-poly(ethylene/propylene) (SEP), polystyrene-b-poly(ethylene/propylene)-b-polystyrene (SEPS), polystyrene-b-poly(ethylene/butylene)-b-polystyrene (SEBS), or polystyrene-b-poly(ethylene-ethylene/propylene)-b-polystyrene (SEEPS), or combinations thereof may be used. Some of such polymers are sold, for example, under the trademarks SEPTON® or KRATON®.

Kraton® D SBS (http://www.kraton.com/Products/Kraton_D_SBS/) The Kraton D SBS family of polymers is versatile with a combination of high strength, wide range of hardness, and low viscosity for easy thermoplastic melt processing or processing in solution. The SBS block copolymers are composed of blocks of styrene and butadiene. It is the material of choice for footwear and the modification of bitumen/asphalt. It is also very useful in pressure sensitive adhesives, hot melt spray diaper adhesives, construction adhesives, impact modification of styrenics, thermoformed clear rigid packaging, and compounds.

Kraton® D SIS (http://www.kraton.com/Products/Kraton_D_SIS/) The Kraton D SIS family of polymers are high performance thermoplastic elastomers with a combination of high strength, low hardness and low viscosity for easy thermoplastic processing as a melt or in solution. SIS polymers are based on styrene and isoprene and are the lowest hardness and lowest viscosity of all the styrenic block copolymers. They are ideally suited for formulating pressure sensitive adhesives (packaging tape, labels, etc.), hot melt spray diaper adhesives, elastic films, and many other innovative applications.

Kraton® FG (http://www.kraton.com/Products/Kraton_FG/) Kraton FG polymers are SEBS polymers with maleic anhydride (MA) grafted onto the rubber midblock. The commercial Kraton FG polymers have 1.0 to 1.7 wt. % MA grafted onto the block copolymer. The MA grafting improves the adhesion to nylon, polyester, ethylene vinyl alcohol, aluminum, steel, glass, and many other substrates. The FG polymers are very efficient impact modifiers in nylon and polyesters for making super tough engineering thermoplastic materials.

Developmental Products

(http://www.kraton.com/Products/Developmental_Products/)

Kraton Polymers is committed to continuous innovation and subsequently is frequently introducing new polymers. These polymers are being specifically designed for new applications and new property sets that cannot be achieved with existing commercial block copolymers.

The Kraton® A polymer series are hydrogenated block copolymers which have styrene copolymerized with ethylene/butylene in the midblock (S-(EB/S)-S).

The Kraton® S polymer series are unsaturated block copolymers that have isoprene and butadiene copolymerized in the midblock (S-(I/B)-S).

The Kraton® ERS polymers have an enhanced ethylene/butylene rubber midblock which is more compatible with polypropylene.

In addition to Kaydol® oil, other plasticizers particularly preferred for use in practicing the present invention are well known in the art, they include rubber processing oils such as paraffinic and naphthenic petroleum oils, highly refined aromatic-free paraffinic and naphthenic food and technical grade white petroleum mineral oils, and synthetic liquid oligomers of polybutene, polypropene, polyterpene, etc. The synthetic series process oils are high viscosity oligomers which are permanently fluid liquid nonolefins, isoparaffins or paraffins of moderate to high molecular weight.”

The high viscosity triblock and branched copolymers: SEEPS, SEBS, SEPS, (SEB).sub.n, and (SEP).sub.n can be measured under varying conditions of weight percent solution concentrations in toluene. The most preferred and useful triblock and branched copolymers selected have Brookfield Viscosity values ranging from about 1,800 cps to about 80,000 cps and higher when measured at 20 weight percent solution in toluene at 25.degree. C., about 4,000 cps to about 40,000 cps and higher when measured at 25 weight percent solids solution in toluene. Typical examples of Brookfield Viscosity values for branched copolymers (SEB).sub.n and (SEP).sub.n at 25 weight percent solids solution in toluene at 25.degree. C. can range from about 3,500 cps to about 30,000 cps and higher; more typically, about 9,000 cps and higher. Other preferred and acceptable triblock and branched copolymers can exhibit viscosities (as measured with a Brookfield model RVT viscometer at 25.degree. C.) at 10 weight percent solution in toluene of about 400 cps and higher and at 15 weight percent solution in toluene of about 5,600 cps and higher. Other acceptable triblock and branched copolymers can exhibit about 8,000 to about 20,000 cps at 20 weight percent solids solution in toluene at 25.degree. C. Examples of most preferred high viscosity triblock and branched copolymers can have Brookfield viscosities at 5 weight percent solution in toluene at 30.degree. C. of from about 40 to about 50 cps and higher. While less preferred polymers can have a solution viscosity at 10 weight percent solution in toluene at 30.degree. C. of about 59 cps and higher.

The high viscosity triblock, radial, star-shaped, and multi-arm copolymers of the invention can have a broad range of styrene end block to ethylene and butylene center block ratio of about 20:80 or less to about 40:60 or higher. Examples of high viscosity triblock copolymers that can be utilized to achieve one or more of the novel properties of the present invention are styrene-ethylene-butylene-styrene block copolymers (SEBS) available from Shell Chemical Company and Pecten Chemical Company (divisions of Shell Oil Company) under trade designations Kraton G 1651, Kraton G 1654X, Kraton G 4600, Kraton G 4609 and the like. Shell Technical Bulletin SC:1393-92 gives solution viscosity as measured with a Brookfield model RVT viscometer at 25.degree. C. for Kraton G 1654X at 10% weight in toluene of approximately 400 cps and at 15% weight in toluene of approximately 5,600 cps. Shell publication SC:68-79 gives solution viscosity at 25.degree. C. for Kraton G 1651 at 20 weight percent in toluene of approximately 2,000 cps. When measured at 5 weight percent solution in toluene at 30.degree. C., the solution viscosity of Kraton G 1651 is about 40. Examples of high viscosity SEBS triblock copolymers includes Kuraray's SEBS 8006 which exhibits a solution viscosity at 5 weight percent at 30.degree. C. of about 51 cps. Kuraray's 4055 SEEPS (styrene-ethylene/ethylene-propylene-styrene) block polymer made from hydrogenated styrene isoprene/butadiene block copolymer or more specifically made from hydrogenated styrene block polymer with 2-methyl-1,3-butadiene and 1,3-butadiene which exhibits a viscosity at 5 weight percent solution in toluene at 30.degree. C. of about 90 mPa-S, at 10 weight percent about 5800 mPa-S. Kuraray's 2006 SEPS polymer exhibits a viscosity at 20 weight percent solution in toluene at 30.degree. C. of about 78,000 cps, at 5 weight percent of about 27 mPa-S, at 10 weight percent of about 1220 mPa-S, and at 20 weight percent 78,000 cps. Kuraray SEPS 2005 polymer exhibits a viscosity at 5 weight percent solution in toluene at 30.degree. C. of about 28 mPa-S, at 10 weight percent of about 1200 mPa-S, and at 20 weight percent 76,000 cps. Other grades of SEBS, SEPS, (SEB).sub.n, (SEP).sub.n polymers can also be utilized in the present invention provided such polymers exhibits the required high viscosity. Such SEBS polymers include (high viscosity) Kraton G 1855X which has a Specific Gravity of 0.92, Brookfield Viscosity of a 25 weight percent solids solution in toluene at 25.degree. C. of about 40,000 cps or about 8,000 to about 20,000 cps at a 20 weight percent solids solution in toluene at 25.degree. C.

The styrene to ethylene and butylene (S:EB) weight ratios for the Shell designated polymers can have a low range of 20:80 or less. Although the typical ratio values for Kraton G 1651, 4600, and 4609 are approximately about 33:67 and for Kraton G 1855X approximately about 27:73, Kraton G 1654X (a lower molecular weight version of Kraton G 1651 with somewhat lower physical properties such as lower solution and melt viscosity) is approximately about 31:69, these ratios can vary broadly from the typical product specification values. In the case of Kuraray's SEBS polymer 8006 the S:EB weight ratio is about 35:65. In the case of Kuraray's 2005, 2006, and 4055 the and S:EEP weight ratios are 20, 35 and 30 respectively. Much like S:EB ratios of SEBS and (SEB).sub.n, the S:EP ratios of very high viscosity SEPS, (SEP).sub.n copolymers are expected to be about the same and can vary broadly. The S:EB, S:EP weight ratios of high viscosity SEBS, SEPS, (SEB).sub.n, and (SEP).sub.n useful in forming the gel compositions of the invention can range from lower than about 20:80 to above about 40:60 and higher. More specifically, the values can be 19:81, 20:80, 21:79, 22:78, 23:77, 24:76, 25:75, 26:74, 27:73, 28:72, 29:71, 30:70, 31:69, 32:68, 33:67, 34:66, 35:65, 36:64, 37:63, 38:62, 39:61, 40:60, 41:59, 42:58, 43:57, 44:65, 45:55, 46:54, 47:53, 48:52, 49:51, 50:50, 51:49 and etc. Other ratio values of less than 19:81 or higher than 51:49 are also possible. Broadly, the styrene block to elastomeric block ratio of the high viscosity triblock, radial, star-shaped, and multi-arm copolymers of the invention is about 20:80 to about 40:60 or higher, less broadly about 31:69 to about 40:60, preferably about 32:68 to about 38:62, more preferably about 32:68 to about 36:64, particularly more preferably about 32:68 to about 34:66, especially more preferably about 33:67 to about 36:64, and most preferably about 33:67. In accordance with the present invention, triblock copolymers such as Kraton G 1654X having ratios of 31:69 or higher can be used and do exhibit about the same physical properties in many respects to Kraton G 1651 while Kraton G 1654X with ratios below 31:69 may also be use, but they are less preferred due to their decrease in the desirable properties of the final gel.

Other polymers and copolymers (in major or minor amounts) can be selectively melt blended with one or more of the high viscosity polymers as mentioned above without substantially decreasing the desired properties; these (III) polymers include (SBS) styrene-butadiene-styrene block copolymers, (SIS) styrene-isoprene-styrene block copolymers, (low styrene content SEBS) styrene-ethylene-butylene-styrene block copolymers, (SEP) styrene-ethylene-propylene block copolymers, (SEPS) styrene-ethylene-propylene-styrene block copolymers, (SB).sub.n styrene-butadiene and (SEB).sub.n, (SEBS).sub.n, (SEP).sub.n, (SI).sub.n styrene-isoprene multi-arm, branched or star-shaped copolymers and the like. Still, other (III) polymers include homopolymers which can be utilized in minor amounts; these include: polystyrene, polybutylene, polyethylene, polypropylene and the like.”

Applicant has discovered that the copolymer gel filler of the present invention provides a much more realistic feel than the silicone gel filler of the prior art prostheses. Furthermore, in using the gel copolymer filler, the drape and rheological properties of the present invention are more consistent with natural human tissue.

Referring again to FIG. 2, adhered to the rear skin (3) of the breast prosthesis is a lightweight fabric (6) made of polyester and Lycra®. Alternatively, Nylon® could also be employed. The fabric may be printed with a logo and an attractive pattern if desired. The fabric (6) serves several purposes. First of all, it serves as an aesthetic enhancement, and as a means to remove moisture from perspiration through wicking. The fabric also provides a structure to the rear surface of the breast prosthesis that serves to hold the shape of the form. The fabric could be adhered to the rear skin by any conventional technique, preferably a thermal bonding process.

The uniqueness of the breast prosthesis of the present invention is that its copolymer gel filler is a thermoplastic elastomer that can be reconfigured or reshaped by subjecting the prosthesis to heating, followed by reshaping, and then allowing the prosthesis to cool until it retains the new shape. The breast prosthesis can be heated by any conventional source that would not be detrimental to the overall structure. Such sources would include a conventional hair dryer, submersion into a pool of hot water, or any type of oven set to an appropriate temperature. The copolymer gel filled breast prosthesis is a considerable advantage over silicone gel filled breast prostheses with respect to customization. As discussed earlier, the silicone gel filled breast prostheses of the prior art are thermosetting and cannot be reshaped once they have been vulcanized. It is highly desirable to have a breast form that conforms to the shape of the residual tissue or to a natural breast formation, both for quality of appearance and more importantly comfort. The tissue of a post operative chest cavity is highly sensitive for a considerable amount of time following the surgery so any addition to comfort level could greatly increase quality of life for the patient.

Since the copolymer gel filler of the present invention is mostly comprised of mineral oil which is an abundant commodity and much cheaper than silicone, the cost of manufacturing the breast prosthesis of the present invention is drastically reduced.

I claim:

1. A lightweight breast prosthesis comprising:
a front skin and a rear skin having their peripheral edges secured together to define a cavity therein, a filler comprising a mixture of thermoplastic copolymer, plasticizing oil and optionally glass, ceramic or plastic hollow microspheres completely filling said cavity, said skins and filler having an initial configuration approximate the shape of a natural breast, whereby said skins and thermoplastic filler are adapted to be reshaped and customized to the individual wearer by subjecting the prosthesis to heating, reshaping, and then cooling until the prosthesis retains its new shape.

2. A lightweight breast prosthesis as defined in claim 1, wherein said front and rear skins are comprised of polyurethane film.

3. A lightweight breast prosthesis as defined in claim 2, wherein said peripheral edges are secured together by an adhesive or by heat application.

4. A lightweight breast prosthesis as defined in claim 1, wherein said thermoplastic copolymer comprises a hydrogenated styrenic block copolymer comprised of a SEPS type: polystyrene-b-poly(ethylene/propylene)-b-polystyrene, or of a SEBS type: polystyrene-b-poly(ethylene/butylene), or of a SEEPS type: polystyrene-b-poly(ethylene-ethylene/propylene)-b-polystyrene, or of a mixture of any of the copolymers above.

5. A lightweight breast prosthesis as defined in claim 1, wherein a lightweight fabric is thermally adhered to a rear surface of the rear skin adapted to remove moisture from perspiration through wicking and hold the shape of the prosthesis.

6. A lightweight breast prosthesis as defined in claim 5, wherein said lightweight fabric comprises a polyester and Lycra® or polyester and Nylon®.

7. A lightweight breast prosthesis as defined in claim 5, wherein said lightweight fabric includes a logo imprinted thereon.

8. A lightweight breast prosthesis as defined in any one of claims 1-7, wherein said mixture is comprised of 80% to 98% mineral oil, 2% to 12% copolymer, and 0% to 15% glass micro spheres.

9. A lightweight breast prosthesis as defined in any one of claims 1-7, wherein said mixture is comprised of approximately 89% mineral oil, 3.5% SEPS, 0.2% SEEPS, and 7.3% glass micro spheres

10. A lightweight breast prosthesis as defined in any one of claims 1-7, wherein said mixture is comprised of approximately 89% Kaydol® oil, 3.5% Septon® 2006 SEPS, 0.2% Septon® 4033 SEEPS, and 7.3% glass micro spheres.
